(19) United States
(12) Patent Application Publication
(10) Pub. No.: US 2026/0187545 A1
(43) Pub. Date: Jul. 2, 2026

(54) SYSTEMS AND METHODS FOR COORDINATING EXECUTION OF AN ENSEMBLE OF MACHINE LEARNING MODELS

(71) Applicant: Nucs AI Inc., Dover, DE (US)

(72) Inventor: Farid Yagubbayli, Berlin (DE)

(73) Assignee: Nucs AI Inc., Dover, DE (US)

(21) Appl. No.: 19/005,041

(22) Filed: Dec. 30, 2024

Related U.S. Application Data
(60) Provisional application No. 63/678,601, filed on Aug. 2, 2024.

(51) Int. Cl. G06N 20/20; G06T 7/00; G06V 10/764; G06V 10/88

(52) U.S. Cl. CPC G06N 20/20; G06T 7/0012; G06V 10/764; G06V 10/88; G06T 2207/10081; G06T 2207/10104; G06T 2207/30096; G06V 2201/032; G06V 2201/07

(57) ABSTRACT

System for coordinating execution of an ensemble of machine learning models to determine anatomical structures to target during cancer treatment are described herein. In examples, the systems can coordinate execution of multiple machine learning models based on different types of three-dimensional images of a patient. These images can include positron emission tomography (PET) images, computed tomography (CT) images, and/or other similar images. The outputs of the models can be correlated with one another to quantify locations and volumes of tumor lesions within the patient. In some examples, a tumor stage can be determined based on the quantification of the tumor lesions. This information can then be used to determine one or more optimal treatment plans for the patient.

### TECHNICAL FIELD

The present disclosure relates generally to systems and methods for diagnosing different types of cancer in patients and, in some non-limiting embodiments, to systems and method for coordinating execution of an ensemble of machine learning models to determine anatomical structures involved when diagnosing and scoring different types of cancer in patients.

### BACKGROUND

Recent developments in diagnosing the progression of cancer have led to improvements in selecting and implementing treatment plans for patients that, likewise, have led to improvements in patient outcomes. For example, as imaging technologies improve, increasingly higher-resolution images can be generated by imaging devices to allow clinicians to inspect certain portions within the patients' bodies. But despite these improvements, it can still be difficult for clinicians (e.g., oncologic doctors) to identify types of cancer using these images, particularly in early stages where such cancers are generally imperceptible to clinicians. As a result, these types of cancer can be diagnosed later, which can in turn result in the selection of treatment plans that are under-inclusive and target only larger, easier to identify instances of cancer within the patients' bodies. Further, types of cancer that can be targeted with specific therapies can become ineffective as these cancers go undetected and undergo mutations that are resistant to the specific therapies.

### SUMMARY

For the aforementioned reasons, there is a need for systems and methods that improve the identification, localization, and quantification of tumor lesions when using medical imaging techniques.

By virtue of the implementation of the techniques described herein, systems can be configured to detect true positive and true negative tumor lesions (e.g., prostate cancer lesions and corresponding tumor lesions that are associated with prostate cancer metastasis) that can be identified to initially guide clinician review of the state of the patient represented by PET/CT images as described herein. In examples involving prostate cancer, the techniques described herein can be configured to detect true positive and true negative tumor lesions based on PSMA-PET images (also referred to as “functional images”) and corresponding CT images. The system can then localize the detected tumor lesions relative to the anatomical structures they affect in the patient (e.g., using metrics such as TNM scores as described herein), quantify the tumor lesions, and provide such quantification as an output identifying prostate cancer lesions on PSMA-PET/CT images to guide initial review of the state of the patient by the clinician treating the patient, as well as disease staging and (in many cases) treatment planning. The techniques described allow for more precise localization, quantification, and scoring of tumor lesions throughout the body of a patient (including specific anatomical structures such as a prostate of a patient, a liver of a patient, and/or the like). For example, implementation of the techniques described herein can allow systems to identify, localize, and quantify tumor lesions that would otherwise go undetected using conventional techniques (e.g., because such tumor lesions are imperceptible to clinicians or would otherwise be identified as noise by conventional systems). This can allow for earlier staging and treatment of types of cancer and selection of targeted treatments that can, in turn, result in better short- and long-term health outcomes for patients that would otherwise go undiagnosed at earlier stages of cancer progression. The presently-disclosed techniques also improve the determination of tumor, node, metastasis (TNM) scores that can, in turn, allow for faster and more targeted treatment and improvement of health outcomes for patients. And by automating this TNM scoring, the resulting diagnosis for a given patient can be made consistent over conventional methods which relied on clinicians exercising professional judgment based on varying degrees of experience.

Further, by implementing multiple models as part of a pipeline, the systems and methods described herein can be configured to more accurately identify the location of tumor lesions that are associated with given anatomical features of a patient. For example, a first masking model configured to analyze PET images as described herein can be configured to broadly identify tumor lesions throughout the body of a patient. In some instances, the first masking model can be fine tuned to identify and classify tumor lesions of one or more specific anatomical structures (e.g., a prostate and/or the like). A structure-specific model can then be configured to analyze a portion the PET images to identify other types of cancer lesion (e.g., metastasis in the liver that can result from an original organ (e.g., a prostate) being affected with a cancer) that are specific to different anatomical structures where increased precision is desired over the precision available through use of the first masking model. By basing the determination of a TNM stage for the patient on the outputs of the models in the described pipeline, the information used to generate the TNM score can be improved. This can, in turn, allow for improvement in the subsequent targeting of cancer therapies in accordance with the stage of cancer for the patient, allowing for improvements to the health outcome of the patient than would otherwise be achievable using conventional techniques. For example, patients at earlier stages can be identified as candidates for certain treatments that are less-invasive (e.g., targeted use of molecules that have less adverse side effects on patients as opposed to broader, more harsh treatments such as chemotherapy). And for patients at later stages, treatment plans can be developed that involve forgoing chemotherapy where the benefits (e.g., extension of life) are minimal in comparison to the expected reduction in quality of life by implementing such treatment.

In an embodiment, a system for coordinating execution of an ensemble of machine learning models is disclosed. The system can include one or more processors configured to execute a lesion detection model based on functional image data associated with a PET image of a patient. The lesion detection model can be configured to generate a first output including first lesion labels that indicate points within the patient associated with metabolic activity indicative of tumor lesions. In some aspects, the one or more processors can be configured to execute a masking model based on structural image data associated with a CT image of the patient. The masking model can be configured to generate a second output including a plurality of anatomical masks that identify (e.g., segment) volumes within the patient that correspond to anatomical structures. In some aspects, the one or more processors can be configured to correlate a first set of points within the CT image that indicate at least one first lesion based on a correspondence between the PET image (or functional image) and the CT image. The one or more processors can be configured to correlate a second set of points within the CT image that indicate at least one second lesion based on a cropped portion of the PET image and the correspondence between the PET image and the CT image. In some aspects, the one or more processors can be configured to determine a set of anatomical structures that correspond to the at least one first lesion and the at least one second lesion based on locations associated with the first set of points and the second set of points relative to the plurality of anatomical masks.

In some aspects, the one or more processors configured to execute the lesion detection model can be configured to obtain the functional image data based on operation of a Positron Emission Tomography/Computed Tomography (PET/CT) scanner to generate the PET image of the patient. The one or more processors configured to execute the masking model can be configured to obtain the structural image data based on operation of the PET/CT scanner to generate the CT image of the patient. In some aspects, the one or more processors can be further configured to determine an alignment between the PET image and the CT image of the patient. The one or more processors can be configured to determine the correspondence between the PET image and the CT image based on the alignment.

In some aspects, the one or more processors configured to execute the lesion detection model can be configured to execute the lesion detection model to generate the first output, where the first output includes anatomical structure labels indicating a type of anatomical structure. The one or more processors configured to correlate the second set of points can be configured to crop a set of points that have anatomical structure labels corresponding to a first anatomical structure from the functional image data to generate a cropped set of points. The one or more processors can be configured to execute a structure-specific model based on the cropped set of points and a second anatomical mask associated with the first anatomical structure, the structure-specific model configured to generate a third output including second lesion labels that indicate the second set of points. In some aspects, the one or more processors can be further configured to determine that the at least one first lesion of the patient corresponds to a first set of anatomical structures of the patient; and determine a state of the patient based on the at least one first lesion of the patient in the first set of anatomical structures of the patient.

In some aspects, the one or more processors configured to determine the state of the patient can be configured to determine a tumor stage of the at least one first lesion in the first set of anatomical structures. In some aspects, the lesion labels can indicate presence of tumor legions in a prostate of the patient and one or more of a pelvic node, a distant node relative to the prostate of the patient, a bone, or a viscera of the patient. In aspects, the lesion labels can indicate presence of tumor lesions in any individual portion of the body such as, for example, the prostate, the pelvic node, the distant node relative to the prostate, the bone, or the viscera of the patient.

In another embodiment, a method for coordinating execution of an ensemble of machine learning models to determine anatomical structures to target during cancer treatment is disclosed. The method can include executing, by one or more processors, a lesion detection model based on functional image data associated with a PET image of a patient. The lesion detection model can be configured to generate a first output including first lesion labels that indicate points within the patient associated with metabolic activity indicative of tumor lesions. The method can include executing, by the one or more processors, a masking model based on structural image data associated with a CT image of the patient. The masking model can be configured to generate a second output including a plurality of anatomical masks that identify volumes within the patient that correspond to anatomical structures. The method can include correlating, by the one or more processors, a first set of points within the CT image that indicate at least one first lesion based on a correspondence between the PET image and the CT image. In aspects, the method can include correlating, by the one or more processors, a second set of points within the CT image that indicate at least one second lesion based on a cropped portion of the PET image and the correspondence between the PET image and the CT image. In some aspects, the method can include determining, by the one or more processors, a set of anatomical structures that correspond to the at least one first lesion and the at least one second lesion based on locations associated with the first set of points and the second set of points relative to the plurality of anatomical masks.

In some aspects, executing the lesion detection model can include obtaining, by the one or more processors, the functional image data based on operation of a Positron Emission Tomography (PET) scanner to generate the PET image of the patient. Executing the masking model can includes obtaining, by the one or more processors, the structural image data based on operation of a computed tomography (CT) scanner to generate the CT image of the patient.

In some aspects, the method can include determining, by the one or more processors, an alignment between the PET image and the CT image of the patient. In aspects, the method can include determining, by the one or more processors, the correspondence between the PET image and the CT image based on the alignment.

In some aspects, executing the lesion detection model can include executing, by the one or more processors, the lesion detection model to generate the first output, where the first output includes anatomical structure labels indicating a type of anatomical structure. In aspects, correlating the second set of points can include cropping, by the one or more processors, a set of points that have anatomical structure labels corresponding to a first anatomical structure from the functional image data to generate a cropped set of points. In some aspects, the method can include executing, by the one or more processors, a structure-specific model based on the cropped set of points and a second anatomical mask associated with the first anatomical structure, the structure-specific model configured to generate a third output including second lesion labels that indicate the second set of points.

In some aspects, the method can include determining, by the one or more processors, that the at least one first lesion of the patient corresponds to a first set of anatomical structures of the patient. The method can include determining, by the one or more processors, a state of the patient based on the at least one first lesion of the patient in the first set of anatomical structures of the patient. In some aspects, determining the state of the patient can include determining, by the one or more processors, a tumor stage of the at least one first lesion in the first set of anatomical structures. In some aspects, the lesion labels can indicate presence of tumor legions in a prostate of the patient and one or more of: a pelvic node, a distant node relative to the prostate of the patient, a bone, or a viscera of the patient.

In yet another embodiment, a non-transitory computer-readable medium is disclosed. The non-transitory computer-readable medium can store instructions thereon that, when executed by one or more processors, cause the one or more processors to execute a lesion detection model based on functional image data associated with a PET image of a patient. The lesion detection model can be configured to generate a first output including first lesion labels that indicate points within the patient associated with metabolic activity indicative of tumor lesions. In aspects, the instructions can cause the one or more processors to execute a masking model based on structural image data associated with a CT image of the patient, the masking model configured to generate a second output including a plurality of anatomical masks that identify volumes within the patient that correspond to anatomical structures. In aspects, the instructions can cause the one or more processors to correlate a first set of points within the CT image that indicate at least one first lesion based on a correspondence between the PET image and the CT image. In some aspects, the instructions can cause the one or more processors to correlate a second set of points within the CT image that indicate at least one second lesion based on a cropped portion of the PET image and the correspondence between the PET image and the CT image. In aspects, the instructions can cause the one or more processors to determine a set of anatomical structures that correspond to the at least one first lesion and the at least one second lesion based on locations associated with the first set of points and the second set of points relative to the plurality of anatomical masks.

In some aspects, the techniques described herein relate to a non-transitory computer-readable medium, wherein the instructions that cause the one or more processors to execute the lesion detection model cause the one or more processors to: obtain the functional image data based on operation of a Positron Emission Tomography (PET) scanner to generate the PET image of the patient, and wherein the instructions that cause the one or more processors to execute the masking model cause the one or more processors to: obtain the structural image data based on operation of a computed tomography (CT) scanner to generate the CT image of the patient.

In some aspects, the instructions can further cause the one or more processors to determine an alignment between the PET image and the CT image of the patient. The instructions can cause the one or more processors to determine the correspondence between the PET image and the CT image based on the alignment.

In some aspects, the instructions that cause the one or more processors to execute the lesion detection model can cause the one or more processors to execute the lesion detection model to generate the first output, where the first output includes anatomical structure labels indicating a type of anatomical structure. The instructions that cause the one or more processors to correlate the second set of points can cause the one or more processors to crop a set of points that have anatomical structure labels corresponding to a first anatomical structure from the functional image data to generate a cropped set of points. The instructions can cause the one or more processors to execute a structure-specific model based on the cropped set of points and a second anatomical mask associated with the first anatomical structure, the structure-specific model configured to generate a third output including second lesion labels that indicate the second set of points.

In some aspects, the instructions can further cause the one or more processors to determine that the at least one first lesion of the patient corresponds to a first set of anatomical structures of the patient. The instructions can cause the one or more processors to determine a state of the patient based on the at least one first lesion of the patient in the first set of anatomical structures of the patient. In some aspects, the instructions that cause the one or more processors to determine the state of the patient can cause the one or more processors to determine a tumor stage of the at least one first lesion in the first set of anatomical structures.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1A illustrates a diagram of a system for coordinating execution of an ensemble of machine learning models.

FIG. 1B illustrates an ensemble of machine learning models that is configured to analyze PET/CT images of a patient, according to an embodiment.

FIG. 2 illustrates a flow diagram of a process for coordinating execution of an ensemble of machine learning models, according to an embodiment.

FIGS. 3A-3H illustrates an example implementation of the process for of FIG. 2, according to an embodiment.

FIG. 4 illustrates a flow diagram of a process for predicting treatment response, according to an embodiment.

FIG. 5 illustrates an implementation of the process of FIG. 4 when predicting treatment response based on whole body tumor quantification, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are configured to be considered within the scope of the subject matter disclosed herein. Other embodiments can be used and/or other changes can be made without departing from the spirit or scope of the present disclosure. The illustrative embodiments described in the detailed description are not meant to be limiting of the subject matter presented.

Some of the example systems and methods described herein, as well as the techniques they implement, involve coordinating execution of an ensemble of machine learning models to analyze metabolic activity (represented by PET images, SPECT images, or the like, of a patient) at locations or regions within the body of the patient (represented by CT images of the patient). Through coordination of multiple models in an ensemble as described herein, processes for identifying tumor lesions, their locations, and quantification of such lesions, along with subsequent determinations of TNM scores for patients diagnosed with various types of cancer can be improved. This can, in turn, allow for more accurate categorization of these tumor lesions and determination of anatomical structures to be targeted (or not targeted) during treatment, resulting in improved health outcomes for the patient being diagnosed and treated.

In some embodiments, systems can include one or more processors that are configured to execute a lesion detection model (see, e.g., first model 152) based on functional image data associated with a first three-dimensional image of a patient (e.g., a functional image such as a PET image), the lesion detection model configured to generate a first output comprising first lesion labels that indicate points within the patient associated with metabolic activity indicative of tumor lesions. In some implementations, the one or more processors can be configured to execute a masking model (see, e.g., second model 154) based on structural image data associated with a second three-dimensional image (e.g., a CT image) of the patient, the masking model configured to generate a second output comprising a plurality of anatomical masks that segment volumes within the patient corresponding to anatomical structures (e.g., a prostate, liver, and/or the like). While the present disclosure describes the use of functional images such as PET images and other images such as CT images when executing certain operations, it will be understood that such description is not intended to be limiting and that the output of a PET/CT scanner can be processed to generate the PET image and CT images used as described herein. In implementations, the one or more processors can be configured to correlate a first set of points (associated with metabolic activity identified in the PET image as indicative of tumor lesions for certain anatomical structures) at locations within the second three-dimensional image that indicate (e.g., correspond to) at least one first lesion based on a correspondence between the first three-dimensional image and the second three-dimensional image. And in some implementations, the one or more processors can be configured to correlate a second set of points (associated with metabolic activity identified in a particular anatomical structure that is analyzed separately) within the second three-dimensional image that indicate at least one second lesion based on a cropped portion of the first three-dimensional image and the correspondence between the first three-dimensional image and the second three-dimensional image. This cropped portion can be analyzed by a separate model (e.g., a structure-specific model) that is trained and/or fine tuned to analyze the metabolic activity for a given anatomical structure. The one or more processors can also be configured to determine a set of anatomical structures that correspond to the at least one first lesion and the at least one second lesion based on locations associated with the first set of points and the second set of points relative to the plurality of anatomical masks. TNM scores can then be determined based on the anatomical structures where the at least one first lesion and the at least one second lesion are located. By processing the cropped portion using a separate model, the initial lesions that can be obscured by anatomical structures that exhibit physiological metabolic activity in PET scans can be identified. This allows for the enhanced visibility of lesions that might otherwise go undetected. And by isolating the cropped portions of the PET scans, systems described herein can improve the accuracy of analysis and diagnosis of patients (including TNM scores for the patients).

By virtue of implementing the techniques described above, systems can locate and classify, and quantify tumor lesions in a patient with increased accuracy, particularly in targeted anatomical structures, over conventional techniques. In the examples described herein, such an output can be used to identify tumor lesions on PSMA-PET/CT images as well as tumor lesions in other anatomical structures (e.g., the liver of a patient) that are analyzed and quantified through a separately-trained model that is specific to the other anatomical structures to guide treatment planning. As a result, more precise identification, localization, and quantification of tumor lesions locations throughout the body of a patient can be achieved and, in some instances, to increased accuracy when localizing tumor lesions can also be achieved when diagnosing the progression of cancer within a patient. For example, implementation of the techniques described herein can allow for improved determination of volumetric metrics such as, for example, a tumor lesion's volume (e.g., in mL), a degree of tumor PSMA expression represented as a standardized uptake value (SUV), etc. This can allow for improved TNM scoring and result in fewer instances of early-stage cancer going undetected because such tumor lesions are imperceptible would otherwise be identified as noise by conventional systems analyzing three-dimensional images of patients. And this can, in turn, allow for more accurate and precise localization and quantification of tumor lesions not only in a target or suspected anatomical structure (e.g., a prostate) but in other anatomical structures (e.g., a liver), resulting in earlier and more comprehensive treatment of types of cancer and selection of targeted treatments that result in better short- and long-term health outcomes for patients that would otherwise go undiagnosed at earlier stages of cancer progression.

Further, in some cases, as cancers are diagnosed at increasing rates, the ability of clinicians (e.g., radiologists) to keep up with manual review of PET/CT scans to monitor disease progression can be strained. This can lead to burnout or fatigue, and can result in the introduction of a greater amount of human error during this manual review. Where clinician review is still desired or necessary, implementation of the techniques described herein can guide clinicians when performing manual review of PET/CT scans during initial diagnosis and follow-up monitoring, helping them to quickly target areas of interest within these scans for review. Through implementing these techniques, the introduction of human error can be reduced significantly while also allowing clinicians to more quickly focus their review on portions of these scans that are more relevant than others. And in some instances, the implementation of the techniques described herein can augment traditional review of these scans (e.g., through TNM scoring and quantification of tumors on a per-lesion basis, which is not standard practice).

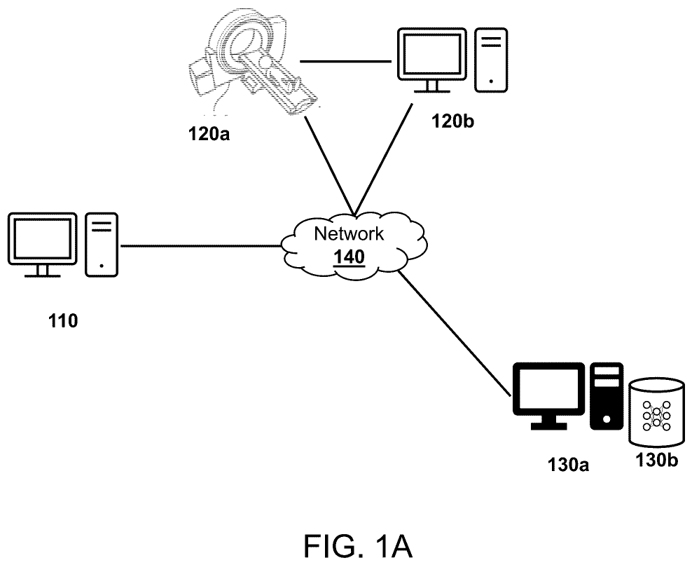

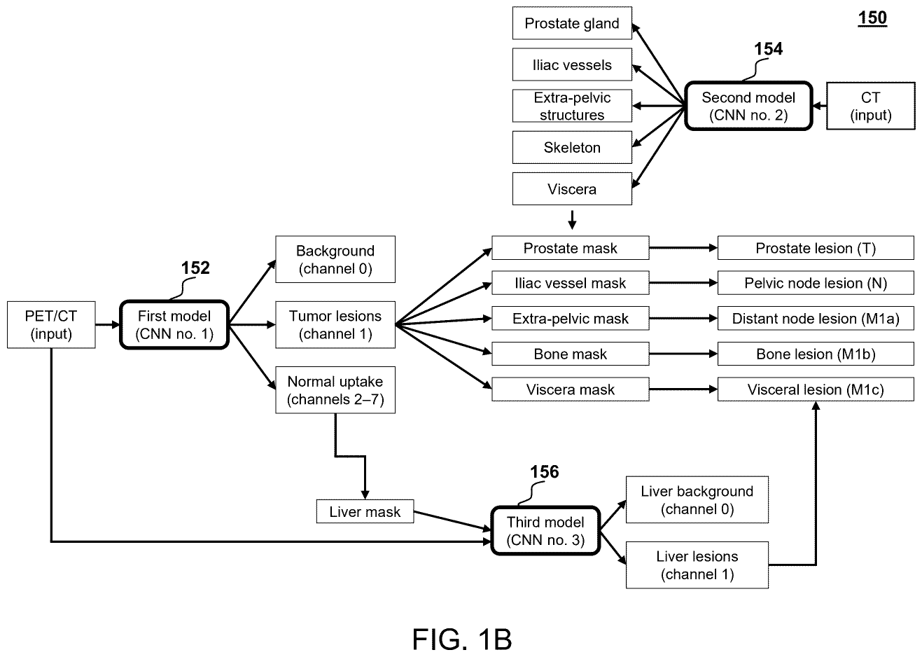

FIG. 1A illustrates components of an environment 100 for coordinating execution of an ensemble of machine learning models, according to an embodiment. The environment 100 can include a client device 110, an imaging device 120a, a clinician workstation 120b, an analytics server 130a, and an analytics database 130b. Various components depicted in FIG. 1A can belong to a treatment clinic involved in diagnosing and treating diseases such as cancers described herein. The environment 100 is not confined to the components described herein and can include additional or other components, not shown for brevity, which are configured to be considered within the scope of the embodiments described herein.

The above-mentioned components can be connected to each other through a network 140. Examples of the network 140 can include, but are not limited to, private or public local-area-networks (LAN), wireless LAN (WLAN) networks, metropolitan area networks (MAN), wide-area networks (WAN), and the Internet. The network 140 can include wired and/or wireless communications according to one or more standards and/or via one or more transport mediums. The communication over the network 140 can be performed in accordance with various communication protocols such as Transmission Control Protocol and Internet Protocol (TCP/IP), User Datagram Protocol (UDP), and IEEE communication protocols. In one example, the network 140 can include wireless communications according to Bluetooth specification sets or another standard or proprietary wireless communication protocol. In another example, the network 140 can also include communications over a cellular network, including, e.g., a GSM (Global System for Mobile Communications), CDMA (Code Division Multiple Access), and EDGE (Enhanced Data for Global Evolution) network.

The client device 110 can be any computing device comprising a processor and non-transitory machine-readable storage capable of executing the various tasks and processes described herein. The client device 110 can employ various processors such as central processing units (CPU) and graphics processing unit (GPU), among others. Non-limiting examples of such computing devices can include workstation computers, laptop computers, server computers, and the like. While the environment 100 includes a single client device 110, the client device 110 can include any number of computing devices operating in a distributed computing environment, such as a cloud environment. In some embodiments, the client device 110 can be associated with a clinician (e.g., an oncologist and/or the like) that is screening and/or treating one or more patients with one or more diseases such as, for example, cancers including prostate cancers and/or the like.

The imaging device 120a can be a diagnostic imaging device or a treatment delivery device. For example, the imaging device 120a can include one or more computed tomography (CT) scanners, positron emission tomography (PET) scanners, a combination PET/CT scanner that is configured to either simultaneously or in rapid succession generate CT images and PET images of a patient, and/or the like. In examples, the imaging device 120a can include SPECT/CT scanners and the analysis described herein can be performed using SPECT images. In some embodiments, the imaging device 120a can be configured to generate one or more CT and/or one or more functional images such as PET images of a patient as described herein. The imaging device 120a can be configured to communicate with various sensors (not explicitly illustrated) that monitor a patient's external biological signals. Non-limiting examples of the sensors can include 3D surfacing mechanisms and optical (or other) sensors configured to monitor the movements by the patient (e.g., in response to respiration by the patient when breathing). In some embodiments, the imaging device 120a can be associated with a clinician that is the same as, or similar to, the clinician associated with the client device 110 and/or any other suitable technician that can operate the imaging device 120a.

The imaging device 120a can be associated with (e.g., interconnected with) a clinician workstation 120b and configured to control operation of the imaging device 120a during operation of the imaging device 120a when generating one or more images (e.g., CT and/or PET images) of a patient. For example, the clinician workstation 120b can be any computing device comprising a processor and non-transitory machine-readable storage capable of executing the various tasks and processes described herein. The clinician workstation 120b can employ various processors such as central processing units (CPU) and graphics processing unit (GPU), among others. Non-limiting examples of such computing devices can include workstation computers, laptop computers, server computers, and the like. In some embodiments, the clinician workstation 120b can be associated with a clinician that is the same as, or similar to, the clinician associated with the client device 110 and/or any other suitable technician that can operate the imaging device 120a.

The analytics server 130a can be any computing device comprising a processor and non-transitory machine-readable storage capable of executing the various tasks and processes described herein. The analytics server 130a can employ various processors such as central processing units (CPU) and graphics processing unit (GPU), among others. Non-limiting examples of such computing devices can include workstation computers, laptop computers, server computers, and the like. While the environment 100 includes a single analytics server 130a, the environment 100 can include any number of analytics servers operating in a distributed computing environment, such as a cloud environment.

The analytics server 130a can generate and display an electronic platform configured to receive and process inputs from clinicians and/or PET/CT images (e.g., generated by the imaging device 120a), and perform one or more of the operations described herein to identify and diagnose the presence of one or more forms of cancer in patients. In some embodiments, the electronic platform generates a graphical user interface (GUI) that is displayed by display devices of the analytics server 130a and/or the client device 110. An example of the electronic platform generated and hosted by the analytics server 130a can include a web-based application or a website configured to be displayed on one or more of the devices of the environment 100.

The analytics database 130b can be any computing device comprising a processor and non-transitory machine-readable storage capable of executing the various tasks and processes described herein. For example, the analytics database 130b can represent various computing devices that contain, retrieve, and/or access data associated with the client device 110, the imaging device 120a and/or the analytics server 130a, such as data associated with current and/or previously monitored patients (e.g., CT images, PET images, SPECT images, tumor locations, and/or the like). For instance, the analytics server 130a can obtain data associated with operation of the imaging device 120a and process the data. The analytics server 130a can then generate a dataset and/or data associated with one or more patients, and use the dataset and/or data to train one or more models and/or model ensembles as described herein.

FIG. 1B illustrates an ensemble 150 of machine learning models that is configured to analyze PET images and CT images of a patient, according to an embodiment. Various components depicted in FIG. 1B can be implemented by one or more devices, alone or in coordination, of an environment that is the same as, or similar to, the environment 100 of FIG. 1A. These devices can include, for example, the analytics server 130a of FIG. 1A.

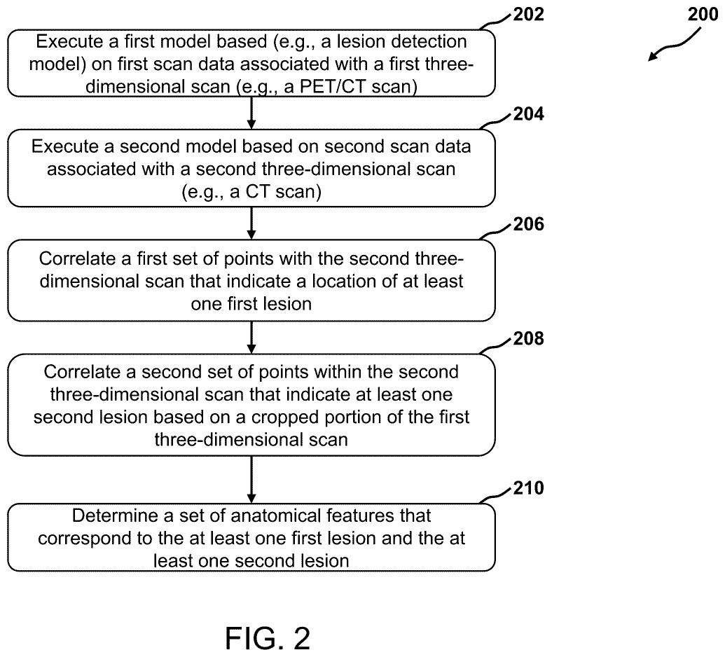

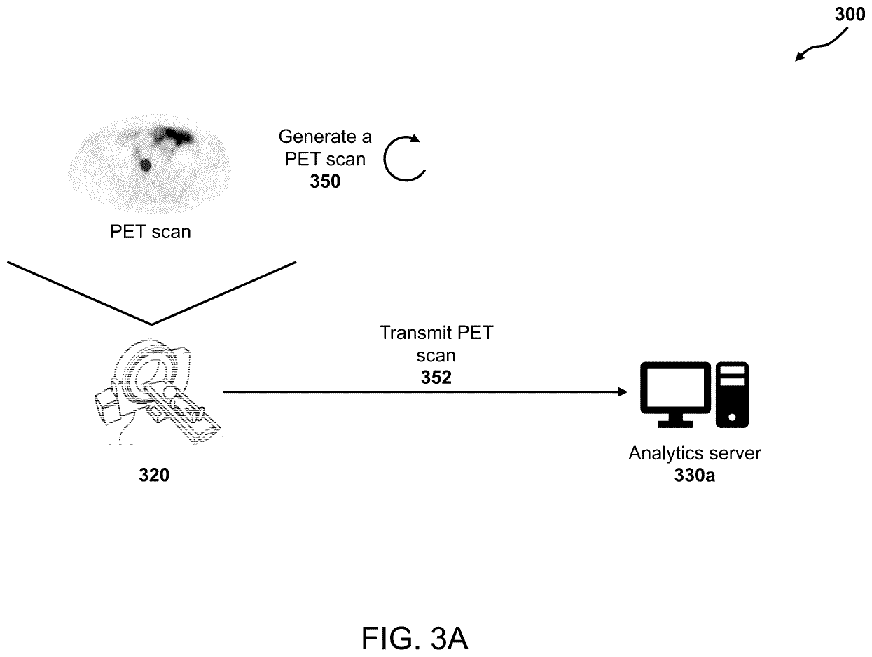

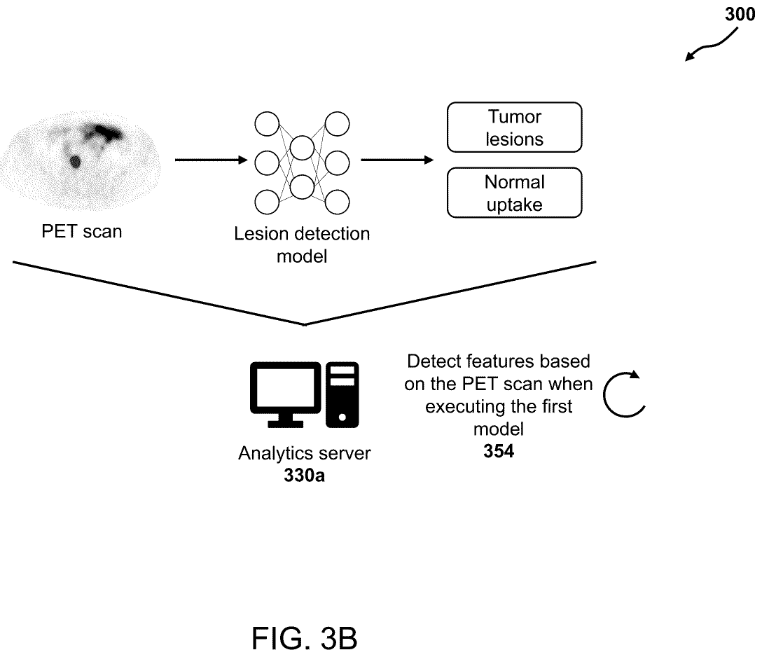

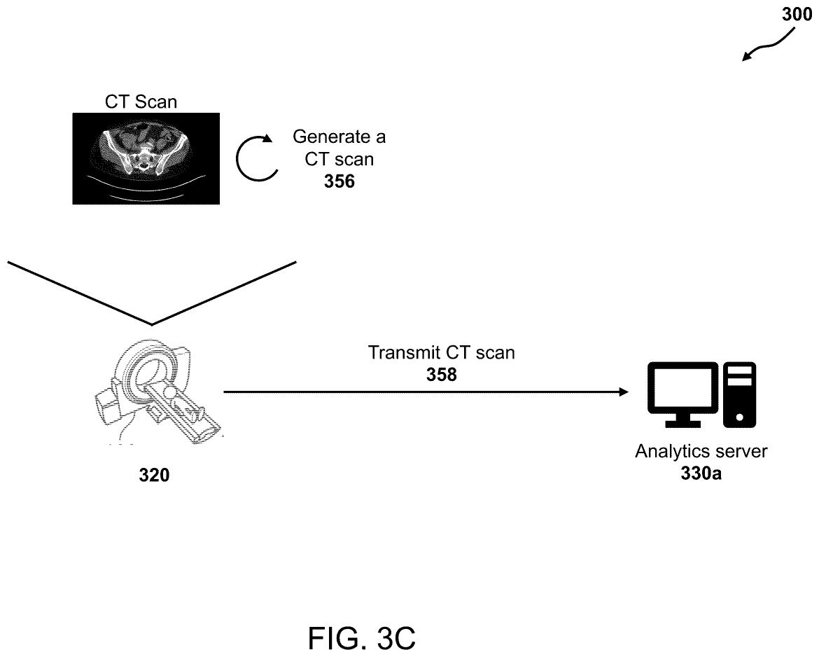

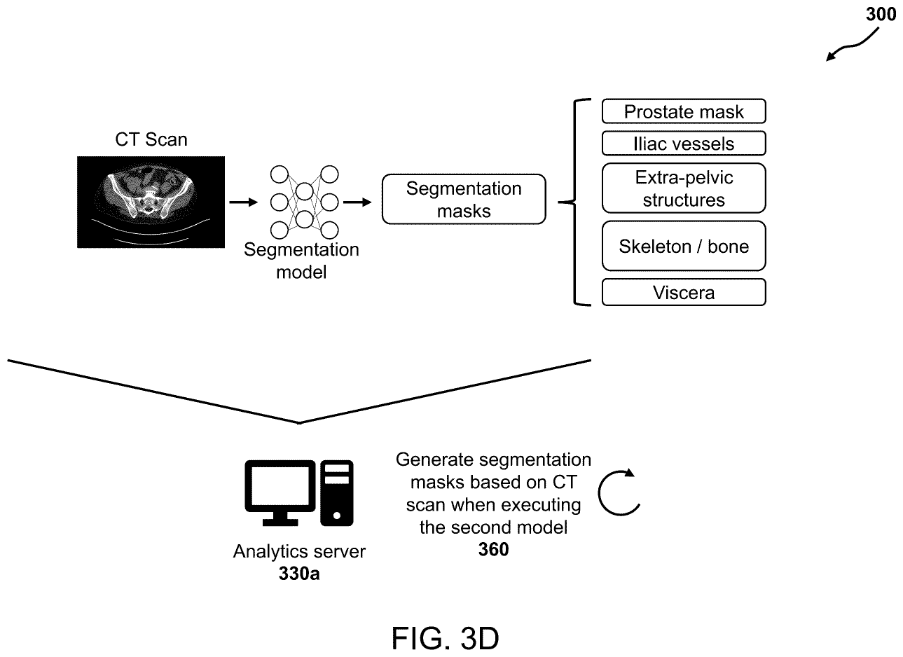

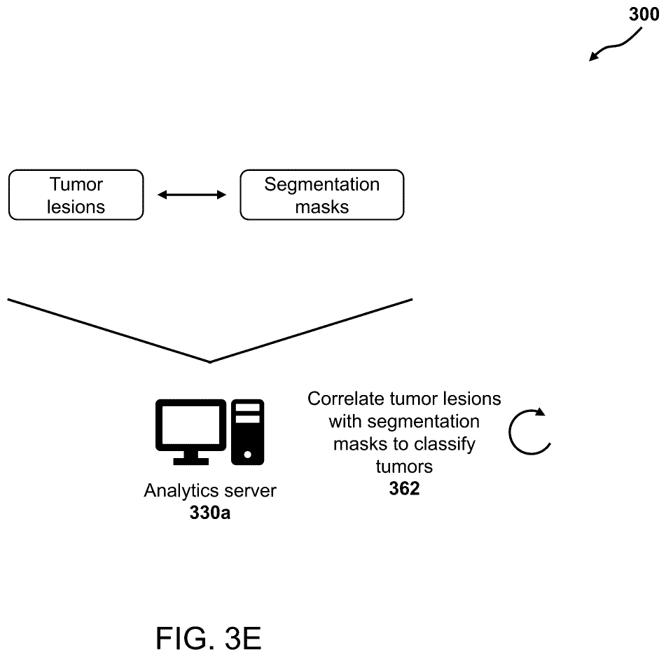

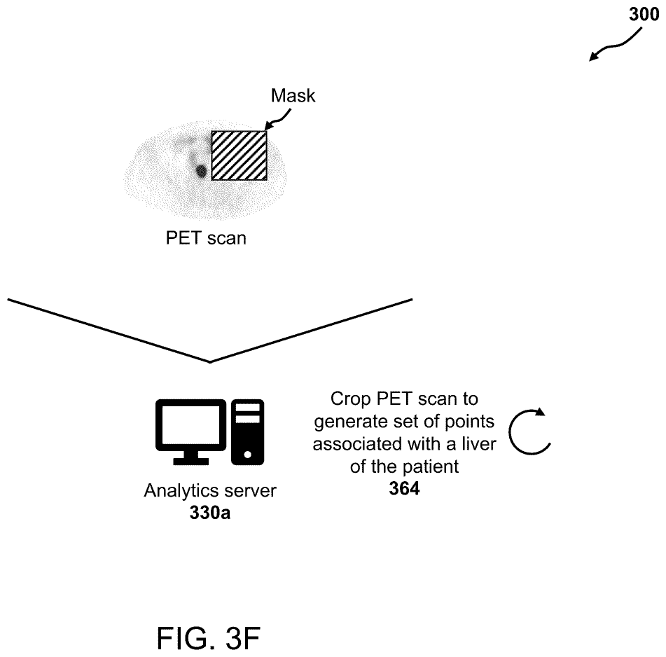

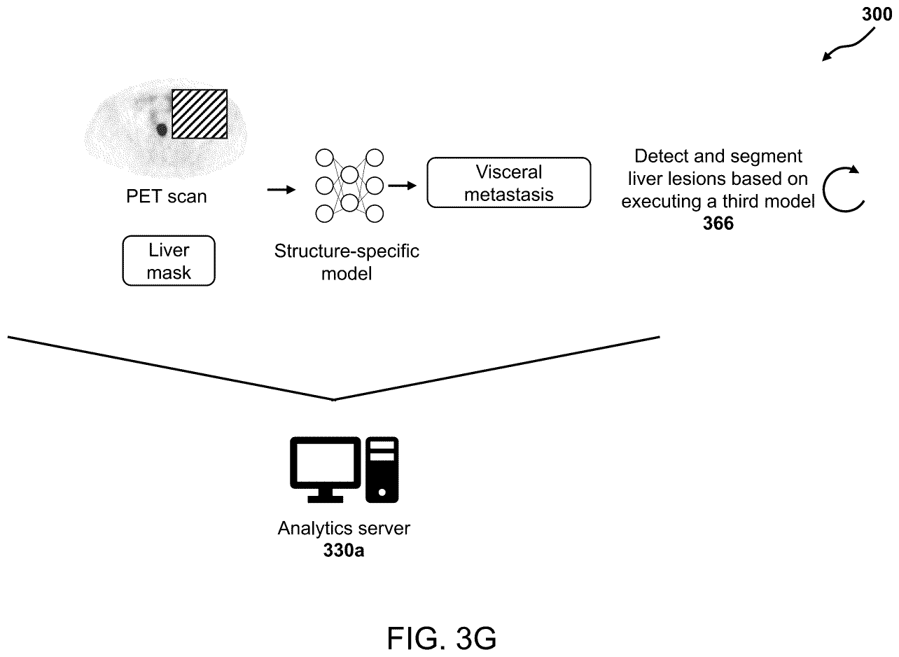

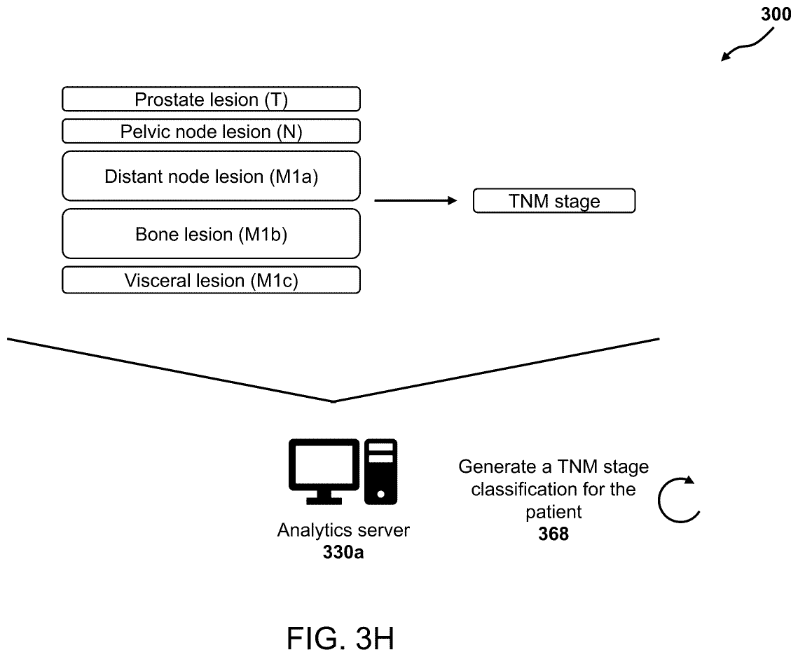

In some embodiments, the analytics server 130a can implement the ensemble 150 where the ensemble 150 includes a first model 152, a second model 154, and a third model 156. For example, the ensemble 150 can include a first model 152 that is a convolutional neural network (CNN). The first model 152 can be the same as, or similar to, a lesion detection model as described with respect to FIG. 2 and/or FIG. 3B. In some embodiments, the first model 152 can be configured to be executed in accordance with the data associated with the one or more types of functional images such as PET images, SPECT images, and/or the like, and generate an output. The output of the first model can represent metabolic activity at a plurality of points (e.g., voxels) located within the body of a patient. This plurality of points can be separated into a plurality of channels. For example, the output can include one or more tumor lesion labels corresponding to a channel that indicate points within the patient associated with metabolic activity indicative of tumor lesions and/or other cancer lesions that form as a result of a metastasis (e.g., from an organ that is initially affected such as a prostate) (referred to as “Tumor lesions, (Channel 1)”). Additionally, or alternatively, the output can include one or more anatomical structure labels. For example, the output can include one or more physiological metabolic structure labels (or “metabolic structure labels”) corresponding to a plurality of normal uptake channels that indicate a type of anatomical structure corresponding to the physiologic metabolic activity represented by the first three-dimensional image (referred to as “Normal Uptake, channels 2-7” that correspond to bladder, kidneys, liver, salivary glands, spleen, and gastrointestinal (GI) tract, respectively). The analytics server 130a can then provide at least a portion of the output of the first model 152 to the third model 156 causing the third model 156 to execute based on the data associated with the normal uptake channels image.

In some embodiments, the ensemble 150 can include a second model 154 that is a convolutional neural network (CNN). The second model 154 can be the same as, or similar to, a masking model as described with respect to FIG. 2 and/or FIG. 3B. In some embodiments, the second model 154 can be configured to generate an output based on execution of the second model 154 in accordance with the data associated with the one or more types of images such as CT images of patient(s). In some embodiments, the second model 154 can be configured to generate the output based on the analytics server 130a providing a CT image as input, causing the second model 154 to execute based on the CT images. In this example, the second model 154 can be configured to generate the output, where the output includes a plurality of anatomical masks corresponding to the anatomical structures of the patient(s). The anatomical masks can include a prostate mask (sometimes referred to as a prostate gland mask), an iliac vessel mask, an extra-pelvic structure mask (e.g., masking anatomical structures such as the aorta, inferior vena cava, duodenum, heart, trachea, esophagus, and/or the like), a skeleton (sometimes referred to as bone) mask, and/or a viscera mask (e.g., masking anatomical structures such as the brain, lungs, adrenal glands, and/or the like).

In some embodiments, the analytics server 130a can determine the location of one or more tumor lesions based on correlations between the tumor lesions identified by the tumor lesion labels output by the first model 152 and the anatomical masks. For example, the analytics server 130a can determine that one or more tumor lesion labels indicative of tumor lesions are correlated with one or more anatomical masks. Where the tumor lesions satisfy a threshold amount (e.g., a threshold quantity, a threshold volume, and/or the like) the analytics server 130a can generate an output indicating that the one or more anatomical structures associated with the respective anatomical masks include one or more tumor lesions. As illustrated in the example of FIG. 1B, the output can indicate one or more prostate lesions (referred to as “Prostate lesion (T)”), pelvic node lesions (referred to as “Pelvic node lesion (N)”), distant node lesions (referred to as “Distant node lesion (M1a)”), bone lesions (referred to as “Bone lesion (M1b)”), and visceral lesions (referred to as “Visceral lesion (M1c)”). The output can then be used by the analytics server 130a when determining a TNM score for the patient. In examples, the analytics server 130a can compare the points of the second three-dimensional image correlated with the metabolic activity indicative of one or more tumor lesions and evaluate the overall tumor size and extent, nodal involvement, and metastasis of the tumor lesions of the patient. In some embodiments, the analytics server 130a can then then determine a stage value from 0-IV, with higher values indicating more advanced progression of the disease, guiding treatment decisions and prognosis.

In some embodiments, the analytics server 130a can update the output indicating that the one or more anatomical structures include one or more tumor lesions based on the output of the third model 156. In some embodiments, the third model 156 can be the same as, or similar to, the structure-specific model as described with respect to FIG. 2 and/or FIG. 3G. Where the output of the third model 156 indicates one or more tumor lesions in a specific anatomical structure (e.g., the liver of the patient) or set of anatomical structures, the analytics server 130a can update the output indicating the anatomical structures including tumor lesions (generated based on the correlations between the output of the first model 152 and the output of the second model 154) to include an indication that the one or more specific anatomical structures associated with the respective anatomical masks include one or more tumor lesions. In some examples, the analytics server 130a can implement the third model 156 such that the third model 156 separately processes the portions of the PET image that is cropped before being processed by the first model 152. This can allow the analytics server 130a to forgo analysis of the specific anatomical structure (that is cropped) using the first model 152 trained to analyze larger portions of the patient, and implement the third model 156, where the third model 156 is fine-tuned relative for the specific anatomical structures to identify tumor lesions in the specific anatomical structures. And by virtue of this separate analysis, computational resources of the analytics server 130a can be more efficiently directed to analyze the specific anatomical structure and provide more accurate outputs where the specific anatomical structure is an anatomical structure of interest but not suspected of being associated with origination of one or more initial tumor lesions.

In some embodiments, an analytics server that implements the ensemble 150 can generate a graphical user interface (GUI) to indicate locations of the one or more tumor lesions that were identified and/or a TNM score that was determined by the analytics server. For example, the analytics server can update the PET images and/or the CT images initially obtained to include the indication of the locations of the one or more tumor lesions. The analytics server can then provide data associated with the GUI to a client device (e.g., that is the same as, or similar to, the client device 110 of FIG. 1A) to cause the client device to generate the GUI. As a result, when the GUI is generated to assist a clinician such as a radiologist reviewing the PET/CT images of the patient, the clinician can more quickly identify portions of interest within the PET/CT images when diagnosing or monitoring disease progression. This can lead reduce the chances of the clinician experiencing burnout or fatigue, and reduce the chance that human error is introduced during manual review of the PET/CT images. And in some instances, the implementation of the techniques described herein can augment traditional review of these scans (e.g., through quantification of tumors on a per-lesion basis, which is not standard practice).

Referring to FIG. 2, illustrated is a flow diagram of a process 200 for coordinating execution of an ensemble of machine learning models to determine tumor lesions to target during cancer treatment. The process 200 includes operations 202-210. However, other embodiments can include additional or alternative operations or can omit one or more operations altogether. The process 200 is described as being executed by an analytics server, which can be the same as, or similar to, the analytics server 130a described in FIG. 1A. However, one or more steps of the process 200 can be executed by any number of computing devices operating in the distributed computing system described in FIG. 1A.

At operation 202, the analytics server can execute a lesion detection model based on functional image data associated with a first three-dimensional image (e.g., a functional image such as a PET image, a SPECT image, and/or the like). For example, the analytics server can execute the lesion detection model based on the functional image data, where the lesion detection model is configured to receive data associated with one or more types of three-dimensional images representing portions of (or the entirety of) a patient. In this example, the lesion detection model can include a convolutional neural network (CNN) configured to receive and process one or more PET images. In some examples, the lesion detection model can include one or more models such as, for example, a vision transformer (ViT), a U-net, a nnU-net, and/or any other similar machine learning model that can be configured to classify points within a three-dimensional volume as being associated with (e.g., including) tumor lesions. In some embodiments, the lesion detection model can be configured to receive data associated with one or more positron emission tomography (PET) images. In some examples, the lesion detection model can be configured (e.g., trained) to receive data associated with one or more other types of images alternative to, or in addition to, the PET images. These images can include one or more Single Photon Emission Computed Tomography (SPECT) images and/or the like.

In some embodiments, the lesion detection model can be configured to generate an output based on execution of the lesion detection model in accordance with the data associated with the first three-dimensional image. For example, the lesion detection model can be configured to generate the output based on (e.g., in response to) receiving the functional image data. In this example, the lesion detection model can be configured to generate the output, where the output is includes one or more lesion labels (e.g., first lesion labels) that indicate points within the patient associated with metabolic activity indicative of tumor lesions and/or other types of cancers that, in some cases, are metastasis that form as a result of cancers forming in different anatomical structures. Additionally, or alternatively, the output can include one or more anatomical structure labels. For example, the output can include one or more anatomical structure labels that indicate a type of anatomical structure corresponding to the metabolic activity represented by the first three-dimensional image. In some examples, the one or more anatomical structure labels can indicate that points within the first three-dimensional image correspond to a prostate, a liver, a bone, and/or the like.

In some embodiments, the analytics server can provide the functional image data to the lesion detection model to cause the lesion detection model to execute and generate an output in accordance with the functional image data based on (e.g., in response to) the operation of one or more devices. These devices can include, for example, an imaging device that is the same as, or similar to, the imaging device 120a of FIG. 1A. In one example, the analytics server can receive the functional image data from the imaging device, where the imaging device includes a PET/CT scanner that can be configured to generate PET images and CT images simultaneously or in rapid succession, and the functional image data can include a three-dimensional image of a body of a patient derived from the output of the PET/CT scanner. The functional image data can be generated based on operation of the imaging device when diagnosing suspected and/or progressing types of cancer such as prostate cancer and/or the like.

In some embodiments, the lesion detection model can be trained and/or updated to generate outputs based on the data associated with one or more types of images representing portions of (or the entirety of) a patient. For example, the lesion detection model can be trained and/or updated based on training datasets including training PET images of patients, where points within the PET images that indicate metabolic activity associated with tumor lesions are tagged with the first lesion labels and/or the anatomical structure labels. These points can be tagged with the first lesion labels based on input provided by clinicians when reviewing the training PET images. Training can include iteratively providing data associated with the one or more types of images to the lesion detection model to cause the lesion detection model to generate outputs, comparing the outputs (e.g., the first lesion labels) with predetermined first lesion labels representing a ground truth, and updating weights of the lesion detection model to cause the lesion detection model to output updated sets of first lesion labels based on the difference between the outputs and the ground truth that more closely approximate the first lesion labels representing the ground truth. This training and/or updating can be repeated until the lesion detection model converges (e.g., where the difference between the outputs of the lesion detection model and the ground truth satisfy a difference threshold).

In some embodiments, the lesion detection model can be trained and/or updated based on training datasets including training PET/CT images of the patients, where points within the PET/CT images that indicate metabolic activity associated with tumor lesions are tagged with the first lesion labels and/or the anatomical structure labels. By jointly training the lesion detection model using PET/CT images similar to as described above (e.g., by providing the PET/CT images to the lesion detection model, comparing the output of the model to a ground truth, and iteratively updating the weights of the model until the model converges) the analytics server can improve the overall operation of the lesion detection model when operating at inference on only PET images.

At operation 204, the analytics server can execute a masking model based on structural image data associated with a second three-dimensional image (e.g., a structural images such as a CT image). For example, the analytics server can execute a masking model based on the structural image data, where the masking model is configured to receive data associated with structural images representing portions of (or the entirety of) a patient. In this example, the masking model can be configured to receive data associated with one or more structural images such as CT images. In examples, the masking model can include a CNN that is the same as, or similar to, the CNN of the lesion detection model. In some examples, the masking model can include one or more models such as, for example, a ViT, a U-net, a nnU-Net, and/or any other similar machine learning model that are the same as, or similar to, the lesion detection model.

In some embodiments, the masking model can be configured to generate an output based on execution of the masking model in accordance with the structural image data. For example, the masking model can be configured to generate the output based on (e.g., in response to) receiving the structural image data. In this example, the masking model can be configured to generate the output, where the output includes one or more anatomical masks that indicate points within the patient corresponding to volumes that match anatomical structures. For example, the output can include one or more anatomical masks indicating a volume corresponding to a prostate, a liver, and/or any other similar anatomical structure. In some examples, the one or more anatomical structure labels can indicate a prostate, a liver, a colon, and/or the like.

In some embodiments, the analytics server can provide the structural image data to the masking model to cause the masking model to execute and generate an output in accordance with the structural image data. For example, the analytics server can receive the structural image data based on (e.g., in response to) the operation of one or more devices. These devices can include, for example, an imaging device that is the same as, or similar to, the imaging device 120a of FIG. 1A. In one example, the analytics server can receive or derive the structural image data based on the output of the imaging device, where the imaging device includes a PET/CT scanner, and the structural image data can include a three-dimensional image of a body of the patient that has the one or more types of cancer. The structural image data can be generated based on operation of the imaging device when diagnosing suspected and/or progressing types of cancers such as prostate cancer and/or the like.

In some embodiments, the masking model can be trained and/or updated to generate outputs based on the data associated with one or more types of images representing portions of (or the entirety of) a patient. For example, the masking model can be trained and/or updated based on training datasets including training CT images of patients, where points within the three-dimensional image associated with anatomical structures such as the prostate, liver, etc., of the patient are tagged to indicate that the points are further associated with an anatomical mask. These points can be tagged as associated with anatomical masks based on input provided by clinicians when reviewing the training CT images. Training can include iteratively providing data associated with the one or more types of images to the masking model to cause the masking model to generate outputs, comparing the outputs (e.g., the anatomical masks) with predetermined anatomical masks representing a ground truth, and updating weights of the masking model to cause the masking model to output updated sets of anatomical masks based on the difference between the outputs and the ground truth that more closely approximate the anatomical masks representing the ground truth. This training and/or updating can be repeated until the masking model converges (e.g., where the difference between the outputs of the masking model and the ground truth satisfy a difference threshold).

At operation 206, the analytics server can correlate a first set of points with the second three-dimensional image (e.g., the structural image) to indicate a location of at least one first lesion within the structural image. For example, the analytics server can correlate a first set of points within the second three-dimensional image with one or more tumor lesions represented by lesion labels from the first three-dimensional image (e.g., the functional image). The correlation can be based on a correspondence between the first three-dimensional image and the second three-dimensional image. In some embodiments, the correlation of the first set of points with the second three-dimensional image can include correlating points within a functional image such as a PET image that are identified (e.g., labeled based on execution of the lesion detection model) as being associated with metabolic activity indicative of one or more tumors with corresponding points within a structural image such as a CT image. For example, the correlation of the first set of points with the second three-dimensional image can include correlating points within the second three-dimensional image of a prostate of a patient with first lesion labels indicating the presence of one or more lesions within the prostate. In examples where a cancer metastasizes (e.g., having originated in a prostate of a patient), the correlation can indicate points within the second three-dimensional image that represent one or more of a regional node lesion, a distant node lesion, a bone lesion, and/or a visceral lesion.

The correspondence can indicate a relationship of relative positions between the first three-dimensional image and the second three-dimensional image. In one example, the analytics server can determine the correspondence based on the analytics server aligning the first three-dimensional image with the second three-dimensional image. The analytics sever can align the images by sequentially scanning the patient using an integrated PET/CT scanner to minimize differences between the images that can be based on, for example, patient movement. The analytics server can then execute one or more operations involved in rigid or non-rigid registration to match the first three-dimensional image with the second three-dimensional image and correct for misalignments due to patient motion or physiological changes. In this example, the analytics server can determine the correspondence based on the results of the operations involving the rigid or non-rigid registration.

At operation 208, the analytics server can correlate a second set of points within the second three-dimensional image that indicate at least one second lesion based on a cropped portion of the first three-dimensional image (e.g., a cropped portion of the functional image). For example, the analytics server can identify points associated with one or more anatomical structures based on the output of the lesion detection model. In this example, the analytics server can identify the points associated with the anatomical structures based on the anatomical structure labels that are included in the output of the lesion detection model. In some embodiments, the analytics server can select one or more subsets of points to be cropped from the first three-dimensional image before being processed by the lesion detection model. For example, when processing the first three-dimensional images (e.g., the PET images) of the patient to identify tumor lesions in the liver, the analytics server can select the points from the first three-dimensional image having anatomical structure labels indicating the points are associated with (e.g., enclosed by and/or in proximity to) the liver of the patient. The analytics server can then identify points in the anatomical structure that correspond to metabolic activity indicative of tumor lesions and correlate those points as the second set of points within the second three-dimensional image.

In some embodiments, the analytics server can process the points associated with one or more anatomical structures to determine one or more lesions within the one or more anatomical structures, including specific anatomical structures (also referred to as physiological metabolic structures). For example, the analytics server can crop the points (e.g., as a set of points) that have anatomical structure labels indicating the points are associated with the anatomical structures from the functional image data (e.g., the PET images). In this example, the analytics serve can generate a cropped set of points based on cropping the points from the functional image data, where the cropped set of points correspond to the physiological metabolic structure(s). In some embodiments, the analytics server can then execute a structure-specific model based on the cropped set of points. For example, the analytics server can execute a structure-specific model that is configured to generate an output in accordance with the points associated with the one or more physiological metabolic structures that are cropped from the first three-dimensional image.

In some embodiments, the structure-specific model can be configured to generate the output based on (e.g., in response to) receiving data associated with the points of the one or more physiological metabolic structure from the first three-dimensional image. For example, the structure-specific model can be configured to generate the output, where the output is associated with (e.g., includes) one or more lesion labels (e.g., second lesion labels) that indicate points within the physiological metabolic structure of the patient that are associated with metabolic activity indicative of tumor lesions. In some examples, where the structure-specific model is configured to process data associated with the points of a physiological metabolic structure such as the liver, the structure-specific model can receive data associated with the liver that is cropped from the first three-dimensional image, and tag a subset of the points of the liver with the second lesion labels based on classification of the points as being associated with tumor lesions. This cropping can occur prior to execution of the lesion detection model on the functional image data, such that the cropped points are not preprocessed or are not otherwise associated with one or more tags indicating metabolic activity indicative of tumor lesions before being processed by the structure-specific model.

In some embodiments, the structure-specific model can be trained and/or updated to generate outputs based on the data associated with one or more types of images representing portions of (or the entirety of) a patient. For example, the structure-specific model can be trained and/or updated based on training datasets including training PET images similar to those described herein, where the training PET images include cropped portions of PET images for individual anatomic structures. Training can include iteratively providing data associated with a physiological metabolic structure to the structure-specific model to cause the structure-specific model to generate outputs, comparing the outputs (e.g., the second lesion labels) with predetermined second lesion labels representing a ground truth, and updating weights of the structure-specific model to cause the structure-specific model to output updated sets of second lesion labels based on the difference between the outputs and the ground truth that more closely approximate the second lesion labels representing the ground truth. This training and/or updating can be repeated until the structure-specific model converges (e.g., where the difference between the outputs of the structure-specific model and the ground truth satisfy a difference threshold).

At operation 210, the analytics server can determine a set of anatomical structures that correspond to the at least one first lesion and the at least one second lesion. For example, the analytics server can determine a set of anatomical structures that correspond to the at least one first lesion, where the at least one first lesion is identified as a target lesion (e.g., to be diagnosed and/or treated). The target anatomical structure can include an anatomical structure (e.g., an organ such as a prostate and/or the like) of a patient that is suspected of including, or is being treated to address, one or more tumor lesions. In some embodiments, the analytics server can determine a set of anatomical structures that correspond to the at least one first lesion. For example, the analytics server can determine a set of anatomical structures that correspond to the at least one first lesion, where the at least one first lesion is a target anatomical structure and the one or more tumor lesions are correlated with a spread of cancer within the target anatomical structure and/or to one or more other anatomical structures of the patient (e.g., the at least one second lesion as described herein). In some examples, the analytics server can determine a set of anatomical structures that correspond to the at least one first lesion, and one or more tumor lesions (e.g., metastasis) that are included in an anatomical structure that is different from the target anatomical structure. In one example, the analytics server can determine that the target anatomical structure is a prostate of a patient, and that one or more tumor lesions are associated with a second anatomical structure such as a liver of the patient.

In some embodiments, the analytics server can determine the set of anatomical structures that correspond to the at least one first lesion and the at least one second lesion based on locations associated with points correlated to the second three-dimensional image. For example, the analytics server can compare the points correlated with the second three-dimensional image with the one or more anatomical masks of the second three-dimensional image. In this example, the analytics server can determine that the points are enclosed in, on the surface of, or in proximity to the anatomical structures associated with the respective anatomical masks. The analytics server can then determine that the one or more anatomical structures that have the one or more lesions.

In one example, the analytics server can determine that the at least one first lesion of the patient is correlated with (e.g., corresponds to) a first anatomical structure. This first anatomical structure can include a target anatomical structure such as a prostate of a patient. The analytics server can then determine that one or more tumor lesions are correlated with (e.g., correspond to) a second anatomical structure of the patient. In examples, the analytics server can determine that the one or more tumor lesions are correlated with one or more structures such as lymph nodes of the pelvis of the patient, one or more bones of the patient, and/or the like. In these examples, the analytics server can determine that the one or more tumor lesions are further associated with the at least one first lesion and that the one or more tumor lesions spread from the at least one first lesion.

In some embodiments, the analytics server can diagnose a state of the patient based on the points correlated with the second three-dimensional image. For example, the analytics server can determine the state of the patient, where the state is represented as through an assessment involving the Tumor, Node, Metastasis (TNM) system. In this example, the analytics server can compare the points of the second three-dimensional image correlated with the metabolic activity indicative of one or more tumor lesions and evaluate the overall tumor size and extent, nodal involvement, and metastasis of the tumor lesions of the patient. In some embodiments, the analytics server can then then determine a stage value from 0-IV, with higher values indicating more advanced diseases, guiding treatment decisions and prognosis.

In some embodiments, the analytics server can generate data that is configured to cause a display device to display a representation of one or more anatomical features. For example, the analytics server can generate data that is configured to cause a display device of a client device to generate a graphical user interface (GUI). The GUI can include a representation of one or more of the anatomical features of the second three-dimensional image correlated with points indicative of tumor lesions. In one example, the GUI can be generated based on a maximum intensity projection (MIP), where voxels associated with intensities of points indicative of tumor lesions are projected to a two-dimensional plane to allow a clinician to identify the location, size, etc., of the tumor lesions. In some examples, voxels associated with the tumor lesions can be viewed individually or in combination when generating the GUI for a particular anatomical structure such as the prostate, liver, bone, and/or the like. Clinicians can also provide input (e.g., via manual drawing with a cursor, hovering with a cursor, and/or the like using an input device of the client device such as a mouse or stylus) that is received by the analytics server to update (e.g., change) at least a portion of a region of interest (ROI). This can include expanding an ROI, reducing the ROI, or adding/removing ROIs. In some embodiments, input provided by clinicians can validate one or more ROIs as hotspots that are suspicious and possibly indicative of one or more tumor lesions.

In some embodiments, the analytics server can generate a report. For example, the analytics server can generate a report indicating one or more metrics that represent the state of the patient, one or more measurements of the one or more tumor lesions. The reports can provide information about the one or more tumor lesions, localization of the one or more tumor lesions (e.g., within the patient or within anatomical structures of the patient), and/or the like. In some embodiments, the analytics server can generate the report based on the correlation of the points within the second two-dimensional image. For example, the analytics server can generate two-dimensional or three-dimensional images of portions of the patient that correspond to the one or more metrics that represent the state of the portion(s) of the patient and include the two-dimensional or three-dimensional images along with one or more indications of the one or more metrics when generating a GUI to be displayed as described herein. In some embodiments, the one or more metrics can identify one or more volumes, signal intensities, locations, and/or TNM values of the one or more tumor lesions.

In some embodiments, the analytics server can train a single model (e.g., an individual CNN with one or more detection heads) to analyze the PET/CT images and identify tumor lesions therein. For example, the analytics server can provide PET/CT images to a CNN to cause the CNN to generate outputs corresponding to the PET/CT images. The outputs can indicate one or more points within the CT images that are indicative of tumor lesions. The analytics server can then compare the outputs of the CNN to a ground truth (e.g., PET/CT images that are annotated by clinicians to indicate where one or more tumor lesions are located within the PET/CT images) and update the weights of the CNN. This process can be iteratively repeated until the CNN converges.

Referring now to FIGS. 3A-3H, an example implementation 300 of the process 200 for coordinating execution of an ensemble of machine learning models to determine anatomical structures to target during cancer treatment. The implementation 300 includes operations 350-368. However, other embodiments can include additional or alternative operations or can omit one or more operations altogether. The implementation 300 is described as being executed by an analytics server, which can be the same as, or similar to, the analytics server 130a described in FIG. 1A. However, one or more steps of the implementation 300 can be executed by any number of computing devices operating in the distributed computing system described in FIG. 1A.

At operation 350, an imaging device 320 can generate a PET image of a patient. For example, the imaging device 320 can generate the PET image to allow an analytics server 330a to locate and quantify the amount of tumor lesions of the patient. In some embodiments, the PET image can indicate one or more points at which metabolic activity of the patient indicates tumor lesions.

At operation 352, the imaging device 320 can transmit the PET image of the patient to the analytics server 330a. This can allow the analytics server 330a to analyze the PET image when locating and quantifying the tumor lesions located in the patient. The result of the analysis can be used by the analytics server 330a to determine one or more treatment plans (e.g., possible treatment plans) for the patient.

At operation 354, the analytics server 330a can detect features based on the PET image when executing a lesion detection model. In some embodiments, the lesion detection model can be the same as, or similar to, the first model 152 of FIG. 1B and/or the lesion detection model described with respect to FIG. 2. The lesion detection model can be configured to receive the PET image as an input and generate first lesion labels and/or anatomical structure labels for one or more points representing corresponding points of the patient in the PET image that are associated with metabolic activity indicative of tumor lesions. For example, the analytics server 330a can detect features such as one or more points associated with one or more tumor lesions, and/or the like (illustrated as “Tumor lesions”). The analytics server 330a can also detect features such as one or more physiological metabolic structure labels (illustrated as “Normal uptake”). The one or more physiological metabolic structure labels can indicate points within the patient that are associated with a given physiological metabolic structure (e.g., points associated with a urinary bladder, a liver, spleen, and/or the like of the patient) that are associated with metabolic activity indicative of tumor lesions.

At operation 356, the imaging device 320 can generate a CT image of a patient. For example, the imaging device 320 can generate the CT image to allow an analytics server 330a to generate anatomical masks that differentiate between anatomical structures within the CT image of the patient. In some embodiments, the CT image can indicate one or more points that are associated with given anatomical structures such as a prostate gland, iliac vessels, extra-pelvic structures, one or more bones, and a viscera of the patient.

At operation 358, the imaging device 320 can transmit the CT image of the patient to the analytics server 330a. This can allow the analytics server 330a to determine one or more anatomical masks for the anatomical structures of the patient.

At operation 360, the analytics server 330a can generate anatomical masks for the anatomical structures of the patient. For example, the analytics server can generate the anatomical masks for the anatomical structures of the patient based on the analytics server executing a masking model. In some embodiments, the masking model can be the same as, or similar to, the second model 154 of FIG. 1B and/or the masking model described with respect to FIG. 2. The masking model can be configured to receive the CT image as input and generate anatomical masks corresponding to the anatomical structures of the patient as output. In some embodiments, the anatomical masks can include, for example, a prostate mask, an iliac vessel mask, an extra-pelvic structure mask, bone masks, a viscera mask, and/or the like.

At operation 362, the analytics server 330a can correlate one or more tumor lesions with the anatomical masks to classify the tumors of the patient. For example, the analytics server 330a can determine an alignment between the PET image and the CT image. In this example, the analytics server can then add lesion labels to respective points (or groups of points, referred to as voxels) indicating that the respective points are associated with one or more tumor lesions. These labels can be added based on the analytics server 330a matching the points having lesion labels in the PET image with the corresponding points in the CT image.

At operation 364, the analytics server 330a can crop the PET image to generate a set of points associated with a liver of the patient. For example, the analytics server 330a can crop the points having anatomical structure labels that indicate the points are associated with a liver of a patient. At operation 366, the analytics server 330a can then detect and segment tumor lesions (referred to as “liver lesions”) within the liver based on execution of a structure-specific model. In some embodiments, the structure-specific model can be the same as, or similar to, the third model 156 of FIG. 1B and/or the structure-specific model described with respect to FIG. 2. In this example, the structure-specific model can be configured to receive the cropped points from the PET image of the patient and the anatomical mask of the liver from the CT image as input and output indications of one or more points that are associated with tumor lesions within the liver. These tumor lesions can be classified as a visceral lesion.

At operation 368, the analytics server 330a can generate a TNM stage classification for the patient. For example, the analytics server 330a can generate the TNM stage classification based on the points of the CT image that are identified as being correlated with the tumor lesions. As illustrated, the analytics server can generate the TNM stage classification based on one or more prostate lesions, one or more regional node lesion, one or more distant node lesion, one or more bone lesion, and/or one or more visceral lesion that are represented by the points correlated with tumor lesions in the CT image of the patient.

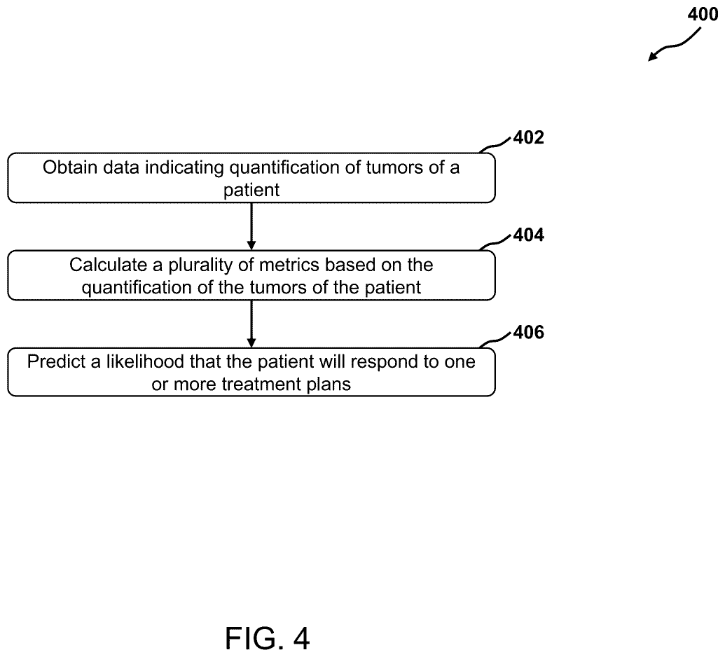

Referring to FIG. 4, illustrated is a flow diagram of a process 400 for predicting treatment response based on whole body tumor detection, localization, and quantification, according to an embodiment. The process 400 includes operations 402-406. However, other embodiments can include additional or alternative operations or can omit one or more operations altogether. The process 400 is described as being executed by an analytics server, which can be the same as, or similar to, the analytics server 130a described in FIG. 1A. However, one or more steps of the process 400 can be executed by any number of computing devices operating in the distributed computing system described in FIG. 1A. In some embodiments, the process 400 can be implemented when screening patients to determine whether one or more targeted therapies satisfy a threshold likelihood of resulting in a positive health outcome for a patient, such as a patient being treated for prostate cancer.

At operation 402, the analytics server can obtain data indicating quantification of tumors of a patient. For example, as described above with respect to FIGS. 2-3H, an analytics server can identify one or more tumor lesions of a patient. While the present disclosure describes the identification of one or more tumor lesions that can originate in a prostate of a patient and metastasize to one or more other anatomical structures such as a liver and/or the like, it will be understood that the present disclosure is not intended to be limiting to such anatomical structures and that other anatomical structures can be analyzed in a similar manner.

In some embodiments, the analytics server can obtain the data indicating the quantification of tumors of a patient based on one or more criteria being satisfied. For example, the analytics server can obtain the data indicating the quantification of the tumors of the patient based on tumor volume and/or a standardized uptake value (SUV) of the patient. The SUV can be measured when a PET image of the patient identifies the presence of a radiopharmaceutical binding to the tumor lesions of the patient (e.g., binds with PSMA that is overexpressed in prostate cancer cells of a patient). In one example, where the SUV of the tumors of the patient in an anatomical structure such as the prostate are greater than the SUV of the tumors of another anatomical structure (e.g., the liver), the analytics server can obtain the data indicating the quantification of the tumors of the patient and determine whether the patient is a candidate for one or more targeted therapies. Additionally, or alternatively, in this example, where a quantity of Prostate-Specific Membrane Antigen (PSMA) of the patient is overexpressed in prostate cancer cells (e.g., expressed beyond a normal amount), the analytics server can determine that the patient is a candidate for both diagnostic imaging and therapy, and obtain the data indicating the quantification of the tumors of the patient.

At operation 404, the analytics server can calculate a plurality of metrics based on the quantification of the tumor lesions of the patient. For example, the analytics server can calculate a plurality of metrics based on the data indicating the quantification of tumor lesions of a patient. The metrics can represent localization of the tumor lesions, volumes of the tumor lesions, uptake of the tumor lesions represented as SUVs, and/or the like.

In some embodiments, the analytics server can determine eligibility for one or more treatments based on the metrics that are based on the quantification of the tumor lesions of the patient. For example, the analytics server can identify one or more tumor lesions as PSMA-positive lesions that include metastatic tumors with PSMA uptake greater than uptake of in functional portions of the liver (parenchyma) of the patient. The analytics server can also identify one or more tumor lesions as PSMA-negative lesions where at least one 1 metastatic lesion is associated with a measurement in a CT image with uptake represented by an SUV value that is less than or equal to that in the liver background.

In some embodiments, the analytics server can determine eligibility for the one or more treatments based on the analytics server determining that one or more target proteins are present in the anatomical structure of the patient. For example, the analytics server can determine that one or more tumor lesions are associated with a PSMA expression that satisfies a threshold value. In this example, the analytics server can determine that the patient is eligible for one or more treatments based on the one or more tumor lesions having the PSMA expression that satisfies a threshold value. In some examples, the analytics server can determine that the patient is not eligible for one or more treatments based on the one or more tumor lesions not satisfying the PSMA expression established by the threshold value.

At operation 406, the analytics server can predict a likelihood that a patient will respond to one or more treatment plans. For example, the analytics server can predict the likelihood that the patient will respond to the one or more treatment plans based on the analytics server calculating the plurality of metrics based on the quantification of the tumor lesions of the patient. In one example, the analytics server can predict the likelihood that the patient will respond to the one or more treatment plans where the patient has one or more tumor lesions that are associated with a PSMA expression that satisfies a threshold value. In this example, the analytics server can predict that it is likely the patient will respond to one or more targeted treatments (e.g., one or more targeted therapies such as 177Lu-PSMA-617 and/or the like). Additionally, or alternatively, the analytics server can predict the likelihood that the patient will respond to the one or more treatment plans where the patient has one or more tumor lesions that are associated with a PSMA expression that does not satisfies a threshold value. In these examples, the analytics server can predict that it is not likely the patient will respond to one or more targeted treatments (e.g., one or more targeted therapies such as 177Lu-PSMA-617 and/or the like) where one or more tumor lesions are associated with a PSMA expression that does not satisfies a threshold value (e.g., are PSMA-negative). In these examples, the analytics server can then determine one or more different therapies such as, for example, chemotherapy and/or the like are appropriate when targeting the tumor lesions.

By virtue of the implementation of the techniques described herein, an analytics server can screen patients for one or more targeted therapies and reduce the chances of therapies being administered that have very little chance of success. For example, where a particular therapy is expected to be ineffective in improving the overall health outcome of the patient, the analytics server can determine that it is not likely the patient will respond appropriately to the treatment plans involving the particular therapy. This can be because, for example, one or more tumor lesions are present that are classified as not satisfying PSMA that satisfies a threshold value) and are not expected to respond to PSMA-targeting therapies that are selected to treat one or more other tumor lesions that express PSMA so as to satisfy the threshold value. In one example, where patients have PSMA-expressing tumor lesions (e.g., tumor lesions that are PSMA positive), the analytics server can determine that other tumor lesions have PSMA uptake lower than the liver parenchyma but do not meet CT criteria to be classified as PSMA negative lesions using conventional techniques. Because these lesions with low PSMA uptake lead to limited efficacy of therapies that can target PSMA positive tumor lesions (e.g., 177Lu-PSMA-617, 177Lu-PSMA-I&T), the analytics server can determine that the targeted have very little (if any) chance of success and determine one or more different treatment plans (e.g., involving the use of more general treatments such as chemotherapy) that can be appropriate for targeting the tumor lesions of the patient.

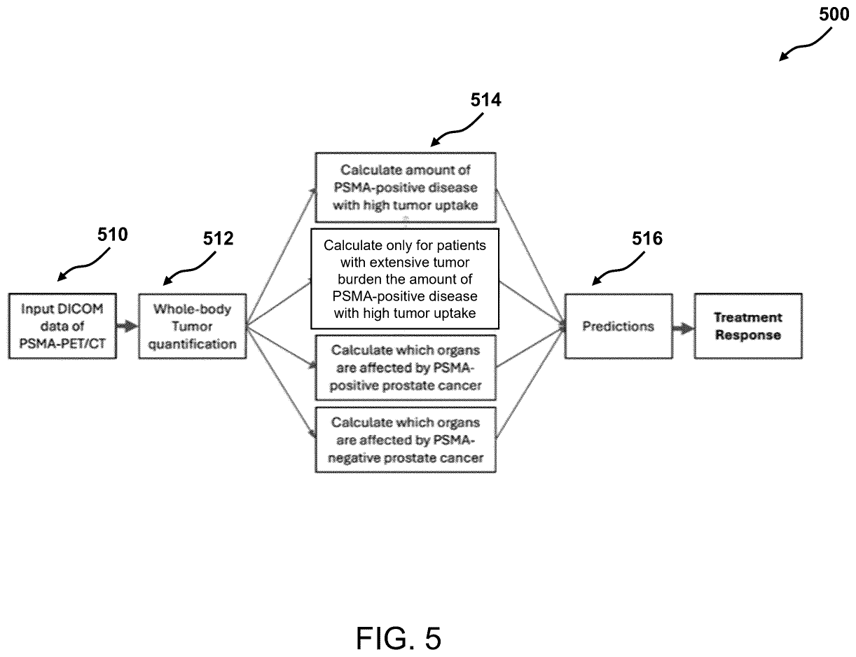

Referring now to FIG. 5, an example implementation 500 of the process 400 for predicting treatment response based on whole body tumor quantification is illustrated. The implementation 500 includes operations 510-516. However, other embodiments can include additional or alternative operations or can omit one or more operations altogether. The implementation 500 is described as being executed by an analytics server, which can be the same as, or similar to, the analytics server 130a described in FIG. 1A. However, one or more steps of the implementation 500 can be executed by any number of computing devices operating in the distributed computing system described in FIG. 1A.

At operation 510, the analytics server can receive data associated with one or more three-dimensional images of a patient. For example, the analytics server can receive data associated with one or more PET images and/or one or more CT images of a patient. The analytics server can then process the one or more PET images and/or the one or more CT images as described herein to identify, localize, and quantify one or more tumor lesions. For example, at operation 512, the analytics server can correlate points of the CT images with corresponding points of the PET images indicative of metabolic activity associated with tumor lesions.

At operation 514, the analytics server can calculate one or more metrics based on the quantification of the one or more tumor lesions of the patient. In the context of prostate cancer diagnosis, the analytics server can calculate a first metric indicating an amount of PSMA-positive tumor lesions. The analytics server can calculate the first metric by determining one or more volumes that are associated with metabolic activity that is determined to indicate PSMA-positive tumor lesions.

In some implementations, the analytics server can calculate a second metric indicating whether the patient has an extensive tumor burden. For example, the analytics server can determine that the tumor lesions represented by the correlation of the PET image with the CT image indicates that an overall amount of a tumor is present or not present in one or more anatomical structures. The correlation can be further determined based on one or more anatomical masks that are associated with the anatomical structures affected by the tumor lesions. In an example, where the analytics server determines that one or more voxels are associated with an anatomical mask of the prostate of the patient, the analytics server can determine that a prostate lesion is present. In another example, where the analytics server determines that one or more voxels are associated with an anatomical mask of the iliac vessels of the patient, the analytics server can determine that a pelvic node lesion is present. In yet another example, where the analytics server determines that one or more voxels are associated with an anatomical mask of the extra-pelvic structures of the patient, the analytics server can determine that a distant node lesion is present. In examples, where the analytics server determines that one or more voxels are associated with an anatomical mask of one or more bones of the patient (e.g., if 30% or more of a bone mask is overlapped with lesion voxels), the analytics server can determine that a bone lesion is present. In some examples, where the analytics server determines that one or more voxels are associated with an anatomical mask of one or more viscera masks of the patient (e.g., if 60% or more of a viscera mask is overlapped with lesion voxels), the analytics server can determine that a viscera lesion is present.

In some embodiments, the analytics server can assign one or more lesions that are not initially correlated with one or more anatomical masks (e.g., are unassigned). For example, where one or more voxels are associated with an anatomical mask of the ribs of the patient (referred to as an extended rib mask) the analytics server can assign the corresponding tumor lesions as a bone lesion. In another example, where one or more voxels are associated with a anatomical mask of the iliac vessels, prostate, bladder, or bone, the analytics server can further analyze the one or more voxels and determine that (1) the lesion is located in proximity to, or in, an extended iliac vessel mask, (2) that the lesion is not located within a bladder mask, (3) that the lesion has a Hounsfield unit (HU) of 200 or less on the CT image, (4) that the lesion does not overlap with the bone mask, and (5) that the lesion is located above an anatomical mask associated with the prostate of the patient. In this example, the analytics server can assign the lesion as a pelvic node lesion. In yet another example, the analytics server can assign one or more lesions that are not initially correlated with one or more anatomical masks based on the analytics server determining that each lesion (1) is on an axis (e.g., a Z-axis) that is common with a anatomical mask that is correlated with an extra pelvic mask, (2) that the lesion has a Hounsfield unit (HU) of 200 or less on the CT image, and (3) that the lesion does not overlap with the bone mask. In this example, the analytics server can assign the lesion as a distant node lesion.

At operation 516, the analytics server can predict a treatment response based on the assignment of the tumor lesions. For example, the analytics server can predict that a targeted treatment will be effective based on combinations of the one or more metrics representing the tumor lesions of the patient. In one example of prostate cancer, where tumor lesions are identified as PSMA-responsive and there are few or no other tumor lesions that are not PSMA-responsive, the analytics server can predict that the targeted treatment will result in an effective treatment response. In another example, the analytics server can predict that a targeted treatment will not be effective based on combinations of the one or more metrics representing the tumor lesions of the patient. In one example, where tumor lesions are identified as not PSMA-responsive, the analytics server can predict that the targeted treatment will not result in an effective treatment response. In this example, the analytics server can indicate that one or more different treatment plans (e.g., the introduction of more general therapies such as chemotherapy) are appropriate for the patient.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for coordinating execution of an ensemble of machine learning models to determine anatomical structures to target during cancer treatment, the system comprising:
one or more processors configured to:
execute a lesion detection model based on functional image data associated with a PET/CT image of a patient, the lesion detection model configured to generate a first output comprising first lesion labels that indicate points within the patient associated with metabolic activity indicative of tumor lesions;
execute a masking model based on structural image data associated with a CT image of the patient, the masking model configured to generate a second output comprising a plurality of anatomical masks that identify volumes within the patient that correspond to anatomical structures;
correlate a first set of points within the CT image that indicate at least one first lesion based on a correspondence between the PET image and the CT image;
correlate a second set of points within the CT image that indicate at least one second lesion based on a cropped portion of the PET image and the correspondence between the PET image and the CT image; and
determine a set of anatomical structures that correspond to the at least one first lesion and the at least one second lesion based on locations associated with the first set of points and the second set of points relative to the plurality of anatomical masks.

2. The system of claim 1, wherein the one or more processors configured to execute the lesion detection model are configured to:
obtain the functional image data based on operation of a Positron Emission Tomography (PET) scanner to generate the PET image of the patient, and
wherein the one or more processors configured to execute the masking model are configured to:
obtain the structural image data based on operation of a computed tomography (CT) scanner to generate the CT image of the patient.

3. The system of claim 1, wherein the one or more processors are further configured to:
determine an alignment between the PET image and the CT image of the patient; and
determine the correspondence between the PET image and the CT image based on the alignment.

4. The system of claim 1, wherein the one or more processors configured to execute the lesion detection model are configured to:
execute the lesion detection model to generate the first output, where the first output comprises anatomical structure labels indicating a type of anatomical structure, and
wherein the one or more processors configured to correlate the second set of points are configured to:
crop a set of points that have anatomical structure labels corresponding to a first anatomical structure from the functional image data to generate a cropped set of points; and
execute a structure-specific model based on the cropped set of points and a second anatomical mask associated with the first anatomical structure, the structure-specific model configured to generate a third output comprising second lesion labels that indicate the second set of points.

5. The system of claim 1, wherein the one or more processors are further configured to:
determine that the at least one first lesion of the patient corresponds to a first set of anatomical structures of the patient; and
determine a state of the patient based on the at least one first lesion of the patient in the first set of anatomical structures of the patient.

6. The system of claim 1, wherein the one or more processors are further configured to:
determine that the at least one first lesion of the patient corresponds to a first set of anatomical structures of the patient, and that the at least one second lesion of the patient corresponds to a second set of anatomical structures of the patient; and
determine a state of the patient based on the at least one first lesion of the patient in the first set of anatomical structures of the patient and the at least one second lesion of the patient in the second set of anatomical structures of the patient.

7. The system of claim 5, wherein the one or more processors configured to determine the state of the patient are configured to:
determine a tumor stage of the at least one first lesion in the first set of anatomical structures.

8. The system of claim 1, wherein the lesion labels indicate presence of tumor legions in a prostate of the patient and one or more of: a pelvic node, a distant node relative to the prostate of the patient, a bone, or a viscera of the patient.

9. A method for coordinating execution of an ensemble of machine learning models to determine anatomical structures to target during cancer treatment, the method comprising:
executing, by one or more processors, a lesion detection model based on functional image data associated with a PET image of a patient, the lesion detection model configured to generate a first output comprising first lesion labels that indicate points within the patient associated with metabolic activity indicative of tumor lesions;
executing, by the one or more processors, a masking model based on structural image data associated with a CT image of the patient, the masking model configured to generate a second output comprising a plurality of anatomical masks that identify volumes within the patient that correspond to anatomical structures;
correlating, by the one or more processors, a first set of points within the CT image that indicate at least one first lesion based on a correspondence between the PET image and the CT image;
correlating, by the one or more processors, a second set of points within the CT image that indicate at least one second lesion based on a cropped portion of the PET image and the correspondence between the PET image and the CT image; and
determining, by the one or more processors, a set of anatomical structures that correspond to the at least one first lesion and the at least one second lesion based on locations associated with the first set of points and the second set of points relative to the plurality of anatomical masks.

10. The method of claim 9, wherein executing the lesion detection model comprises:
obtaining, by the one or more processors, the functional image data based on operation of a Positron Emission Tomography (PET) scanner to generate the PET image of the patient, and
wherein executing the masking model comprises:
obtaining, by the one or more processors, the structural image data based on operation of a computed tomography (CT) scanner to generate the CT image of the patient.

11. The method of claim 9, further comprising:
determining, by the one or more processors, an alignment between the PET image and the CT image of the patient; and
determining, by the one or more processors, the correspondence between the PET image and the CT image based on the alignment.

12. The method of claim 9, wherein executing the lesion detection model comprises:
executing, by the one or more processors, the lesion detection model to generate the first output, where the first output comprises anatomical structure labels indicating a type of anatomical structure, and
wherein correlating the second set of points comprises:
cropping, by the one or more processors, a set of points that have anatomical structure labels corresponding to a first anatomical structure from the functional image data to generate a cropped set of points; and
executing, by the one or more processors, a structure-specific model based on the cropped set of points and a second anatomical mask associated with the first anatomical structure, the structure-specific model configured to generate a third output comprising second lesion labels that indicate the second set of points.

13. The method of claim 9, further comprising:
determining, by the one or more processors, that the at least one first lesion of the patient corresponds to a first set of anatomical structures of the patient; and
determining, by the one or more processors, a state of the patient based on the at least one first lesion of the patient in the first set of anatomical structures of the patient.

14. The method of claim 13, wherein determining the state of the patient comprises:
determining, by the one or more processors, a tumor stage of the at least one first lesion in the first set of anatomical structures.

15. The method of claim 9, wherein the lesion labels indicate presence of tumor legions in a prostate of the patient and one or more of: a pelvic node, a distant node relative to the prostate of the patient, a bone, or a viscera of the patient.

16. A non-transitory computer-readable medium storing instructions thereon that, when executed by one or more processors, cause the one or more processors to:
execute a lesion detection model based on functional image data associated with a PET image of a patient, the lesion detection model configured to generate a first output comprising first lesion labels that indicate points within the patient associated with metabolic activity indicative of tumor lesions;
execute a masking model based on structural image data associated with a CT image of the patient, the masking model configured to generate a second output comprising a plurality of anatomical masks that identify volumes within the patient that correspond to anatomical structures;
correlate a first set of points within the CT image that indicate at least one first lesion based on a correspondence between the PET image and the CT image;
correlate a second set of points within the CT image that indicate at least one second lesion based on a cropped portion of the PET image and the correspondence between the PET image and the CT image; and
determine a set of anatomical structures that correspond to the at least one first lesion and the at least one second lesion based on locations associated with the first set of points and the second set of points relative to the plurality of anatomical masks.

17. The non-transitory computer-readable medium of claim 16, wherein the instructions that cause the one or more processors to execute the lesion detection model cause the one or more processors to:
obtain the functional image data based on operation of a Positron Emission Tomography (PET) scanner to generate the PET image of the patient, and
wherein the instructions that cause the one or more processors to execute the masking model cause the one or more processors to:
obtain the structural image data based on operation of a computed tomography (CT) scanner to generate the CT image of the patient.

18. The non-transitory computer-readable medium of claim 16, wherein the instructions further cause the one or more processors to:
determine an alignment between the PET image and the CT image of the patient; and
determine the correspondence between the PET image and the CT image based on the alignment.

19. The non-transitory computer-readable medium of claim 16, wherein the instructions that cause the one or more processors to execute the lesion detection model cause the one or more processors to:
execute the lesion detection model to generate the first output, where the first output comprises anatomical structure labels indicating a type of anatomical structure, and
wherein the instructions that cause the one or more processors to correlate the second set of points cause the one or more processors to:
crop a set of points that have anatomical structure labels corresponding to a first anatomical structure from the functional image data to generate a cropped set of points; and
execute a structure-specific model based on the cropped set of points and a second anatomical mask associated with the first anatomical structure, the structure-specific model configured to generate a third output comprising second lesion labels that indicate the second set of points.

20. The non-transitory computer-readable medium of claim 16, wherein the instructions further cause the one or more processors to:
determine that the at least one first lesion of the patient corresponds to a first set of anatomical structures of the patient; and
determine a state of the patient based on the at least one first lesion of the patient in the first set of anatomical structures of the patient.

21. The non-transitory computer-readable medium of claim 20, wherein the instructions that cause the one or more processors to determine the state of the patient cause the one or more processors to:
determine a tumor stage of the at least one first lesion in the first set of anatomical structures.
